(19) United States
(12) Patent Application Publication
(10) Pub. No.: US 2007/0191493 A1
(43) Pub. Date: Aug. 16, 2007

(54) Use of n-alkanols as activators of the cftr channel

(76) Inventors: Bernard Verrier, Roquevaire (FR); Brice Marcet, Marseille (FR); Patrick Delmas, Marseille (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/562,085

(22) PCT Filed: Jun. 29, 2004
(86) PCT No.: PCT/FR04/01662

§ 371 Date: Mar. 2, 2007

(30) Foreign Application Priority Data
Jul. 2, 2003 (FR) 0308064

(51) Int. Cl. A61K 31/045

(52) U.S. Cl. 514/724

(57) ABSTRACT

The invention relates to a novel use of n-alkanols as activators of the CFTR (systic fibrosis transmembrane conductance regulator) channel, and to the application of said use to treatments of pathologies in which a dysfunction of said channel can be observed, such as mucoviscidosis.

The present invention relates to a novel use of n-alkanols as CFTR (cystic fibrosis transmembrane conductance regulator) channel activators and to the application of said use to treatments for pathologies in which a dysfunction of said channel is observed, such as cystic fibrosis.

The CFTR protein, located in the apical region of epithelial cells, is a chloride channel controlled by the cAMP and involved in the hydration of fluids secreted by the submucosal glands. A dysfunction of this CFTR channel is responsible for cystic fibrosis, an autosomal recessive genetic disease.

In epithelial cells, water transport and electrolyte transport are associated with an increase in membrane permeabilities for K⁺, Na⁺ and Cl⁻ ions. These water and electrolyte movements are related to the activity of specialized membrane proteins (ion channels, transporters) that have a precise location in the plasma membrane of the cell (apical or mucosal pole; basolateral or serosal pole). The techniques of molecular electrophysiology (patch-clamp) and of ion flux measurement make it possible to study transepithelial ion transport, regulation thereof and pathological deregulation thereof.

A dysfunction of epithelial cells, and in particular that of electrolyte transport, is the cause of many physiopathologies, such as cystic fibrosis (CF) (or mucoviscidosis), which is considered to be an exocrine gland genopathy.

The gene called CF involved in cystic fibrosis has been identified, cloned and located on the long arm of chromosome 7 (Riordan et al., 1989). Cystic fibrosis is the most common autosomal recessive genetic disease in Caucasian populations. In the United States and in most European countries, the frequency of heterozygous carriers of the mutated CF gene is 1 in 20 to 1 in 30, which represents one birth of an affected child in approximately 2500 to 3000. Progress made in the field of medical and biological research has, since the 1960s, brought about considerable progress in the life expectancy of patients suffering from cystic fibrosis, who today live to approximately 30 years old. The CF gene consists of 250000 base pairs defining 27 exons and encodes the CFTR (cystic fibrosis transmembrane conductance regulator) protein, which comprises 1480 amino acids (Riordan et al., 1989). Cystic fibrosis is a canalopathy, i.e. a pathology related to an ion channel dysfunction, insofar as the CFTR protein has been characterized as a chloride channel. At the current time, more than 1300 mutations in the CF gene, which impair the properties and the function of the CFTR channel, have already been reported.

The CFTR protein is expressed in many organs, including the exocrine pancreas, the lungs, the sweat glands, the intestine, the hepatic tissue, the reproductive system, the kidneys and the heart tissue.

The interest given to cystic fibrosis has had considerable consequences in terms of the understanding of the secretory mechanisms of normal epithelial cells. The epithelial cells of the exocrine glands of various organs, such as the intestine, the pancreas or the lungs, control water and salt transport in the tissues. The CFTR protein, which is especially located at the apical pole of epithelial cells, is a low-conductance chloride channel activated by the cAMP pathway.

CFTR is involved in the hydration of fluids secreted by the submucosal glands and is thought to influence the secretion of mucins, which are glycoproteins that contribute in particular to the formation of the bronchial mucus.

In cystic fibrosis, the dysfunction of the CFTR channel affects the cAMP-activated apical secretion of Cl ions. The electrolyte transport, which has become abnormal, causes thickening of the extracellular mucus and thus leads to obstructions in the lumens of the various tissues. These obstructions cause chronic bronchitis due to opportunistic pulmonary bacterial infections, pancreatic and hepatic insufficiencies, abnormally concentrated sudoriparous secretion, and male infertility.

The CFTR protein is a glycoprotein with a molecular weight of 170 kD comprising five domains (Riordan et al., 1989); two transmembrane domains each with 6 transmembrane segments or α-helices (numbered from 1 to 12, each comprising 21 to 22 amino acids), two nucleotide-binding intracellular domains (NBD1 and 2 for nucleotide binding domain) and a large intracellular regulatory domain (R domain).

The regulation of CFTR has been particularly studied. Two complex processes control the activity of the CFTR channel; phosphorylation of the R domain by protein kinases and binding and hydrolysis of ATP to and on the two NBD domains. Dephosphorylation of the CFTR channel results in a loss of activity of the channel until it closes (Tabcharani et al., 1991; Becq et al., 1994).

Several studies have shown that the CFTR protein has, in addition to its chloride channel activity, many other cellular functions that have not yet been elucidated. It is thought to regulate other ion channels, such as the outwardly rectifying chloride channel ORCC (Schwiebert et al., 1995), the epithelial sodium channel ENaC (Quinton et al., 1999) or the calcium-dependent chloride channel CaCC (Wei et al., 1999). It is also thought to have a regulatory activity on ATP release from the inside to the outside of the cell (Schwiebert et al., 1995).

CFTR shares sequence and structural homology with ABC (for “ATP-binding cassette”) transporters which constitute a large family of membrane proteins that are very conserved in evolution. These transporters are involved in the translocation of varied substrates through cell membranes. However, while, in prokaryotes, many transporter/substrate couples have been defined, this information is more rare among eukaryotes. In mammals, there are currently 48 ABC transporters, the dysfunctions of which could be related to a pathology. The P-glycoprotein (or MDR for multidrug resistance) is involved in cytotoxic drug rejection. CFTR controls transepithelial chloride transport and the hydration of mucosal compartments, whereas one of the isoforms of MDR is thought to be more involved in phosphatidylcholine translocation.

Among the around 1300 mutations of the CF gene recorded to date and which cause cystic fibrosis, the one most commonly found is a deletion of three base pairs in a coding region (exon 10) of the CF gene. This mutation corresponds to the deletion, in the protein, of a phenylalanine at position 508 (ΔF508) in the NBD1 domain. The frequency of appearance of this mutation is 70% of the mutated alleles on average in genetic analyses (Tsui et al., 1991), and 50% of patients are homozygous for this mutation. The consequences of this mutation are dramatic because the abnormal protein derived from the transcription of the mutated gene (ΔF508) is no longer capable of performing its functions in chloride transport in the affected epithelial cells. The absence of a chloride current after stimulation of the exocrine gland epithelial cells by cAMP is the main characteristic that shows the presence of an abnormality in the CF gene and in particular of the mutation (ΔF508). The highest density of mutations is found in the two nucleotide-binding domains (NBD1 and NBD2). Seven other important mutations are present with frequencies of greater than 1%. The G551D mutation corresponds to the substitution of a glycine residue (G) at position 551 of the protein with an aspartic acid (D). CF patients carrying this mutant have a severe pathology with pancreatic insufficiency and serious pulmonary disorders (Cutting et al., 1990). The frequency of observation of this mutation reaches 3 to 5% in certain populations of patients suffering from cystic fibrosis. Unlike the ΔF508 deletion, the CFTR protein carrying the G551D mutation is mature and is incorporated into the membrane (Gregory et al., 1991). However, the mutation results in membrane impermeability and stimulation of the cAMP pathway does not open the channel associated with the expression of this mutant (Gregory et al., 1991; Becq et al., 1994). Other mutations, such as R117H, R334W and R347P, appear with low frequencies of 0.8, 0.4 and 0.5%, respectively, and are associated with less severe pathologies (Sheppard et al., 1993). These three mutants express a mature CFTR protein that is glycosylated, allowing its insertion into the membrane. However, the current amplitude, the unit conductance and the probability of opening the channel, which are associated with each of the three mutations, are modified (Sheppard et al., 1993; Becq et al. 1994). The regulation via the cAMP pathway appears, however, to be normal for these three various mutants, including for ΔF508 (Becq. et al., 1994).

Heterozygous carriers of the CF gene, i.e. having one copy of the normal gene and one of the mutated gene, are generally healthy and represent approximately 5% of the Caucasian population. A selective advantage is suggested to explain the relatively high percentage of this mutation in the heterozygous state in the course of evolution. Heterozygous individuals are thought to have been more resistant to epidemics of typhoid fever, of cholera, of tuberculosis or of secretory diarrhea.

However, a correlation between carriers heterozygous for the CF gene and susceptibility to developing various pathologies, such as asthma, nasal polyposis, chronic sinusitis and bronchitis, bronchiectasis, allergic bronchopulmonary aspergillosis or pancreatitis, has been established by certain studies (Griesenbach et al., 1999). Mutations occurring in flanking regions between exon-introns of the CF gene have also been described. For example, 9-, 7- or 5-thymidine polymorphic variants exist between the intron 8 and the exon 9. The 5T polymorphic variant decreases synthesis of the CFTR protein, which incidentally is normal. The combination of the 5T variant on one allele with a mutation of CFTR on the other allele results in congenital agenesis of vas deferens (CAVD), characterized in male patients by secretory infertility with no other conventional cystic fibrosis condition. A certain proportion of sterile men could in fact be carriers of these mutations in the CF gene, without developing cystic fibrosis per se.

This discovery opens up a discussion on the diagnosis and classification of cystic fibrosis, related to the physiological disorders listed in heterozygous individuals. Alongside the cases of conventional cystic fibrosis, the following atypical cases could thus be defined:

in the conventional case, sick homozygous patients (e.g. ΔF508/ΔF508) or composite heterozygous patients (e.g. ΔF508/G551D) exhibit most of the conditions characterized by this disease;

in atypical cases, the patients, composite heterozygotes (ΔF508/5T . . . ) or true heterozygotes, show various conditions: CAVD, asthma, chronic sinusitis, etc., as specified above.

In order to overcome the functional CFTR protein deficiency, both in conventional cases of cystic fibrosis and in atypical cases marked by various conditions (asthma, bronchiectasis, sinusitis, etc.), it is possible to envision pharmacologically activating the wild-type CFTR protein that is still present (heterozygotes), and the mutants, such as ΔF508 or G551D, that are inserted into the membrane but inactive. Despite deficient targeting of the ΔF508 protein into the membranes of epithelial cells affected by cystic fibrosis, several groups have shown that this protein could be functionally present, in small numbers, in the membranes (Dalemans et al., 1991; Drumm et al., 1991; Becq et al., 1994).

Thus, the use of CFTR channel activators, and in particular of CFTR channel openers, can optimize the chances of success of a pharmacotherapy of diseases related to a dysfunction of the CFTR channel.

Despite the progress made in the genetics of cystic fibrosis and in the biology and biochemistry of the CFTR protein, the pharmacology of CFTR channel openers is relatively undeveloped.

Several studies have shown that, in addition to the general agents which are known to activate the CFTR protein via the cAMP pathway, such as forskolin (FSK), it is possible to use other molecules for activating CFTR channels. The mode of action of these activators is still relatively unknown and their effects limited.

Mention may be made of the few families of molecules known today for their CFTR channel activating or opening properties:

Phenylimidazothiazoles (levamisole and bromotetramisole) (Becq et al., 1994). It has been shown that levamisole and bromotetramisole make it possible to control the activity and level of phosphorylation of the CFTR channel. However, these molecules do not appear to be able to act in all cells. In addition, in a transgenic mouse model exhibiting the G551D/G551D mutation, bromotetramisole did not have the expected activating effect.

Benzimidazolones (NSOO4) (Gribkoff et al., 1994). These compounds, derived from the imidazole ring, such as levamisole, can, under certain conditions, and in particular when the CFTR channel has been phosphorylated, open the channel. Benzimidazolones are also, however, activators of many potassium channels (Olesen et al., 1994) and are, consequently, not very specific for the CFTR channel.

Substituted xanthines such as IBMX (3-isobutyl-1-methylxanthine) or theophylline are first known as inhibitors of intracellular phosphodiesterases (cAMP-degrading enzymes), phosphatases and adenosine-binding membrane-receptor antagonists; they also act on intracellular calcium mobilization. Independently of these properties, they are CFTR channel activators (Chappe et al., 1998). The mechanism of action of xanthines on CFTR is still poorly understood, but could involve their binding to the nucleotide-binding domains (NBD1 and NBD2).

Benzo(c)quinolizinium (PCT international application WO 98/05642; Becq et al., 1999) which are more specific for CFTR but can induce adverse effects; specifically, these molecules activate the CFTR channel via a cAMP-independent pathway. However, at the current time, toxicity tests relating to this family of molecules have not yet been carried out and it is not improbable that these molecules may prove to have toxic effects on animals.

Thus, all the treatments currently recommended either lack specificity or cause too many adverse effects.

For this reason, the applicant has given itself the aim of providing medicinal products that specifically activate the CFTR chloride channel, while at the same time not modifying the baseline cAMP level, and that are for use in the treatment of pathologies related to transmembrane ion flux, especially chloride flux conditions, and especially in epithelial cells in humans or animals.

The aim of the present invention is more particularly to provide novel medicinal products that can be used in the context of the treatment of cystic fibrosis, or of cases of “atypical cystic fibrosis” (asthma, chronic sinusitis, bronchiectasis, etc.), or of the prevention or treatment of obstructions of the bronchial tracts or of the digestive (especially pancreatic or intestinal) tracts, or of cardiovascular diseases or else kidney diseases.

Inventors have in fact found, surprisingly, that certain n-alkanols specifically activate the CFTR (cystic fibrosis transmembrane conductance regulator) chloride channel. The activity of the CFTR channel is measured by means of the radioactive iodide (¹²⁵I) efflux technique or of the patch-clamp technique. The order of activation of CFTR by n-alkanols is hexan-1-ol<heptan-1-ol<octan-1-ol<octan-2-ol<decan-1-ol (1 mM).

A subject of the present invention is, consequently, the use of C₆-C₁₀ linear, possibly branched, or cyclic hydrocarbon-chain n-alkanols, for preparing a medicinal product for use in the treatment of pathologies related to CFTR chloride channel (transmembrane chloride flux) disorders, in particular in epithelial cells, in humans or animals.

The fact that the C₆-C₁₀ n-alkanols do not modify the cAMP level in the cells is an advantage for at least two reasons:

this favors a specificity of interaction between the n-alkanols and the CFTR channel;

this makes it possible to prevent nonspecific side effects which can be induced by an increase in cAMP in the cells.

According to an advantageous embodiment of said use, said n-alkanols are linear, possibly branched, hydrocarbon-chain n-alkanols in which the OH group is in the 1-position (primary alcohol) or in the 2-position (secondary alcohol).

According to another advantageous embodiment of said use, said n-alkanols are cyclic hydrocarbon-chain n-alkanols carrying one or more alcohol groups (cyclohexane, for example).

The n-alkanols have, in this application, a certain number of advantages:

no activation by the n-alkanols is detected in control CHO cells that do not express CFTR, whereas the activation of CFTR by the n-alkanols in CHO (Chinese hamster ovary) cells expressing the CFTR channel is blocked by the addition of glibenclamide (100 μM), used to specifically block the CFTR channel;

the n-alkanols do not modify the baseline cAMP level; the n-alkanols thus specifically activate the CFTR channel via a cAMP-independent pathway. The activation of CFTR by the n-alkanols is independent of the potential effect of these molecules on cellular uncoupling;

the n-alkanols act via a protein kinase C-independent mechanism.

Among the n-alkanols, it is especially octanol that has already been proposed in many applications:

1. as anesthetic molecules; it exerts complex effects on biological membranes. A physicochemical theory had been put forward to explain the effector potency of anesthetics. The effectiveness of anesthetics is thought to increase as a function of their solubility in fats and is thought to be a linear function of the octanol-water partition coefficient (Meyer-Overton rule on anesthesia). In fact, the mechanisms involved in the effects of n-alkanols have not been elucidated. However, two general hypotheses have been proposed to explain the effects of n-alkanols on proteins associated with biological membranes:

The first suggests that n-alkanols impair the physical properties of the membrane, which properties are required for the normal functioning of membrane proteins.

The second suggests that n-alkanols bind directly in hydrophobic regions specific for proteins (Mascia et al., 2000).

It is probable that the two mechanisms are involved. However, it is currently the second hypothesis that holds the interest, the first having been contradicted by experiments which show that disturbances in membrane fluidity, engendered by n-alkanols, were mimicked by an increase in body temperature (fever), without the same effects being observed, however, on the electrical activity.

A common characteristic of the action of these molecules is the modulation of the electrical signal that is due to the impairment of the membrane conductance by the ion channels.

2. In the regulation of the Cl⁻ channel receptor for GABA (gamma-aminobutyric acid) (Narahashi et al., 1998; Marszalec et al., 1994; Nakahiro et al., 1991). These receptors are expressed in the central nervous system.

3. As bronchodilators: n-alkanols are involved in relaxation of the smooth muscles of the airways by decreasing in particular the intracellular concentration of calcium ([Ca²⁻]ᵢ) (Sakihara et al., 2002).

4. Action at cell junctions by impairing the conductance of the gap junctions in many tissues, including the epithelial tissue (Weingart et al., 1998); this effect concerns more specifically lipophilic agents, such as long-chain n-alkanols. The molecular mechanism resulting from the cellular uncoupling remains obscure.

5. Alcohols such as octanol, as anti-emulsifying agents, have already been used, in an aerosol, in patients suffering from pulmonary edema (Miller et al., 1973), but their mechanisms of action remain unknown.

The use of certain n-alkanols in the treatment of pathologies related to transmembrane chloride ion flux disorders in epithelial cells, and in particular of cystic fibrosis and of atypical cystic fibroses, have just been found by the inventors.

Specifically, surprisingly, C₆-C₁₀ n-alkanols, in particular nebulized in the bronchi of patients in the form of an aerosol or of nebulized material, activate or potentiate the activity of wild-type CFTR channels or CFTR channels that have mutated but present at the cell membrane, in particular in patients suffering from cystic fibrosis.

The activation of the CFTR channel by n-alkanols could also promote a bronchodilator effect in the smooth muscle fibers of the bronchi and bronchioles, and contribute to improving the respiratory function of patients suffering from cystic fibrosis, along with patients suffering from respiratory insufficiency not related to a cystic fibrosis, such as asthma.

In general, said n-alkanols can be administered parenterally: intradermal, intravenous, intramuscular or subcutaneous administration; intranasally or buccally: aspiration or nebulization by aerosol; orally; sublingually.

Preferably, said n-alkanols are administered in a form suitable for intranasal or buccal administration, so as to obtain direct contact between said n-alkanols and the surface of the bronchopulmonary mucosae. For example, said n-alkanols are provided in a liquid form, for administration in the form of an aerosol or in the form of nebulized material, by means of a nebulization device, of the type such as those used both in the treatment of asthma and in that of cystic fibrosis.

According to another advantageous embodiment of the invention, said n-alkanols are combined with at least one pharmaceutically acceptable carrier appropriate for said intranasal or buccal administration.

According to another advantageous embodiment of the invention, said n-alkanols are preferably administered at a concentration of between 0.001% and 0.1% (v/v), corresponding to a value of between 10 and 1000 ppm (parts per million), i.e. from 10 mg/kg to 1 g/kg.

Besides the arrangements above, the invention also comprises other arrangements which will emerge from the description which follows, which refers to examples of implementation of the use which is the subject of the present invention, and also to the attached drawings in which:

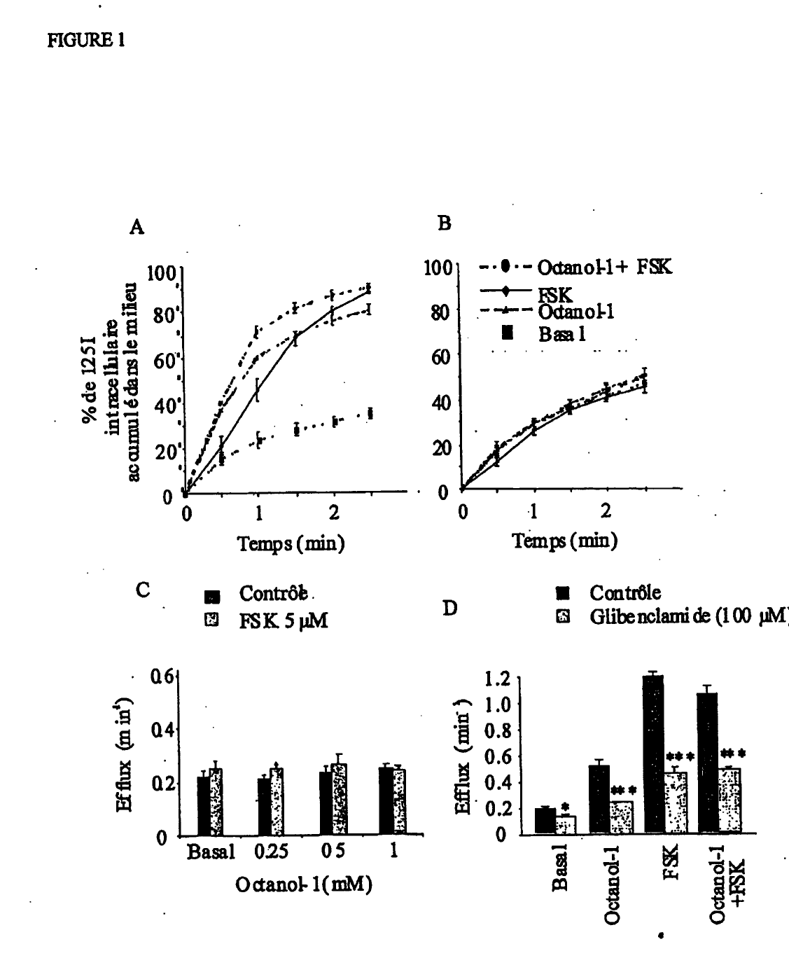

FIG. 1 illustrates: (A) Comparison of the effect of octan-1-ol and of FSK on ¹²⁵I efflux (%, along the y-axis) as a function of time (min, along the x-axis) in CHO-CFTR(+) cells. (B) Effect of octan-1-ol and of FSK on ¹²⁵I efflux (%, along the y-axis) as a function of time (min, along the x-axis) in CHO-CFTR(−) control cells. (C) Effect of octan-1-ol (0.25 to 5 mM) and of FSK (5 μM) on ¹²⁵I efflux (rate of efflux, along the y-axis) in CHO-CFTR(−) control cells. (D) Effect of the specific inhibition of CFTR with 100 μM of glibenclamide on ¹²⁵I efflux (rate of efflux in min⁻¹, along the y-axis) stimulated by octan-1-ol, FSK or octan-1-ol and FSK, in CHO-CFTR(+) cells;

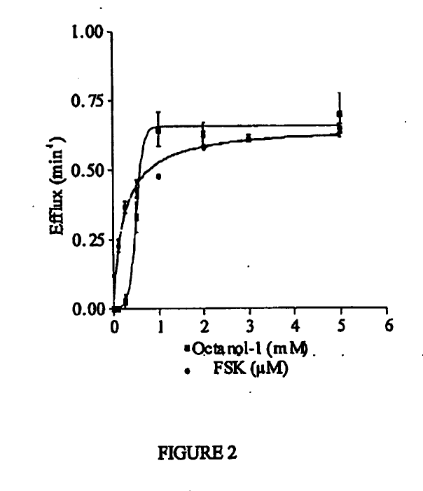

FIG. 2 illustrates the effect of increasing doses (along the x-axis) of FSK or of octan-1-ol on ¹²⁵I efflux (rate of efflux in min⁻¹, along the y-axis) in CHO-CFTR(+) cells;

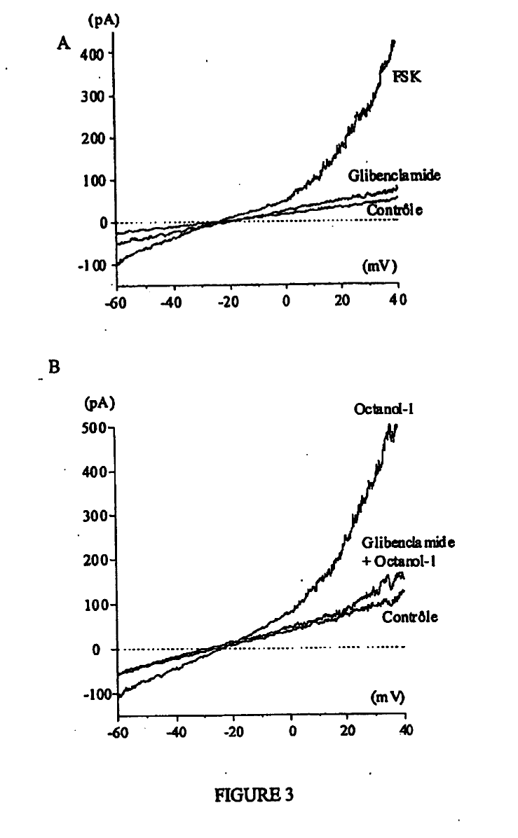

FIG. 3 illustrates: (A) Effect of FSK (1 μM) in the presence or absence of glibenclamide (100 μM) on the current recorded in the whole cell configuration, representing the activation of the CFTR channel. (B) Effect of octan-1-ol (1 mM) on the current induced by the activation of CFTR, in the presence or absence of glibenclamide (100 μM) for specifically inhibiting the CFTR channel. The recording is carried out in CHO-CFTR(+) cells, n=4;

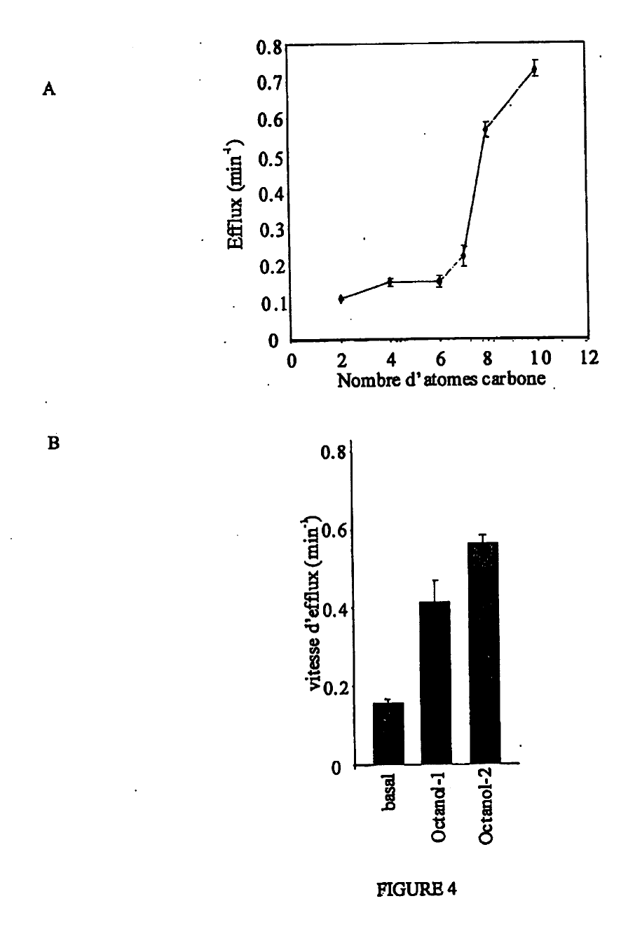

FIG. 4 illustrates: (A) Effect of the length of the hydrocarbon chain of the n-alkanols (along the x-axis) in the activation of ¹²⁵I efflux (rate of efflux in min⁻¹, along the y-axis). (B) Effect of octan-2-ol on the activation of ¹²⁵I efflux (rate of efflux in min⁻¹, along the y-axis);

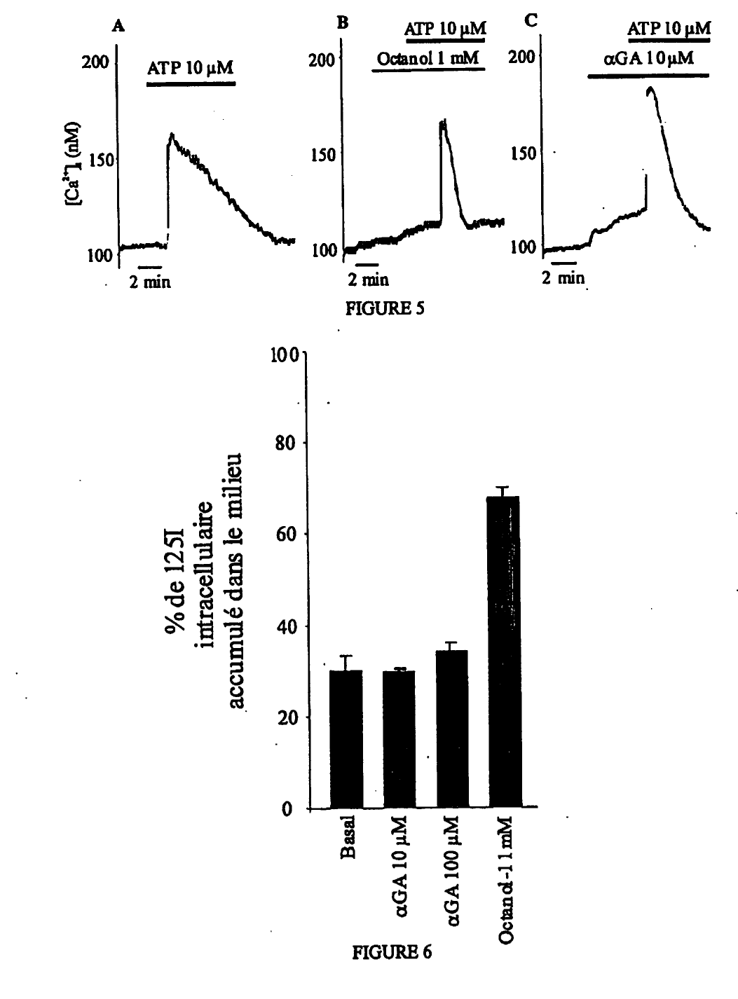

FIG. 5 illustrates the effect of octan-1-ol (1 mM) and of 18-alpha glycerrhetinic acid (α-GA 10 μM) on the calcium response induced by an ATP stimulation that involves intercellular communication;

FIG. 6 illustrates the effect of cellular uncoupling in the activation of ¹²⁵I efflux (%, along the y-axis) by application of 18-alpha glycerrhetinic acid (α-GA 10 and 100 μM) (n=12), in comparison with the effect of octan-1-ol;

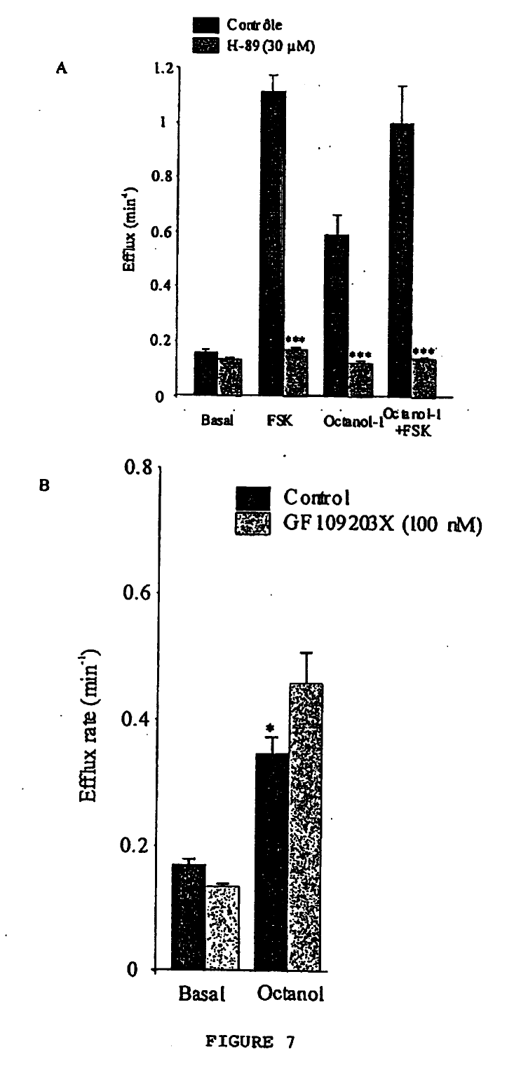

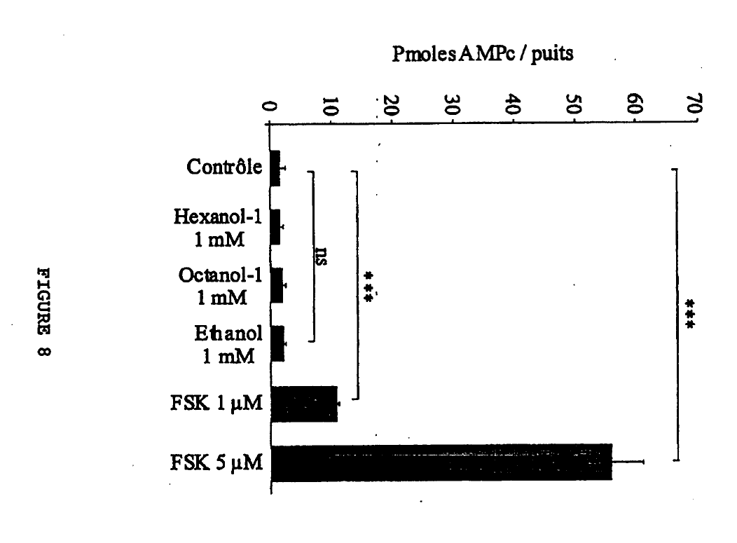

FIG. 7 illustrates: (A) Effect of the inhibition of protein kinase A by H-89 (30 μM, 30 min) on the activation of ¹²⁵I efflux (rate of efflux in min⁻¹, along the y-axis) induced by octan-1-ol (1 mM), FSK (1 μM) or an octan-1-ol+FSK costimulation. (B) Effect of the inhibition of protein kinase C (GF109203X, 100 nM, 30 min) on the activation of ¹²⁵I efflux (rate of efflux in min⁻¹, along the y-axis) induced by octan-1-ol (1 mM);

FIG. 8 illustrates the effect of n-alkanols on the total intracellular cAMP level in comparison with the baseline level and with an FSK stimulation (5 μM);

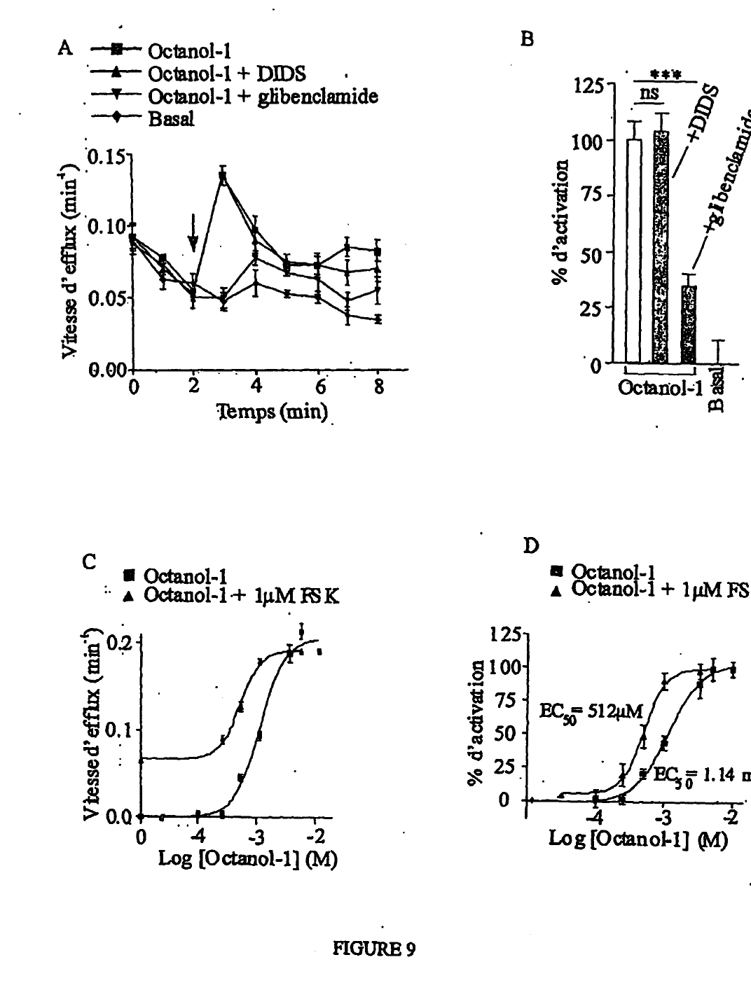

FIG. 9 illustrates: (A) Effect of octan-1-ol (1 mM) on iodide efflux (min⁻¹) as a function of time (min) in human epithelial cells of bronchial origin, Calu-3, with (n=8) or without pretreatment with glibenclamide (100 μM, 1 hour) (n=8), and with (n=8) or without pretreatment with DIDS (500 μM, 1 hour) (n=8). The arrow represents the moment at which the octan-1-ol (1 mM) is added, with or without glibenclamide and with or without DIDS. (B) The maximum effect of octan-1-ol is normalized to 100%. Effect of the treatment with DIDS (500 μM, 1 hour) or glibenclamide (100 μM, 1 hour) on the maximum octan-1-ol response, the baseline level is indicated (t test: *** P<0.001; ns: not significant). (C) Effect of increasing doses (along the x-axis) of octan-1-ol in the presence or absence of FSK (1 μM) on ¹²⁵I efflux (rate of efflux in min⁻¹, along the y-axis) in Calu-3 cells. (D) Dose-response curve for octan-1-ol (n=8 for each concentration tested) in the presence or absence of FSK (1 μM), represented as % of the maximum activation obtained for 10 mM of octan-1-ol. The half-effect concentration (EC₅₀) for octan-1-ol is 512 μM in the presence of FSK (1 μM) and 1.14 mM in the absence of FSK (1 μM);

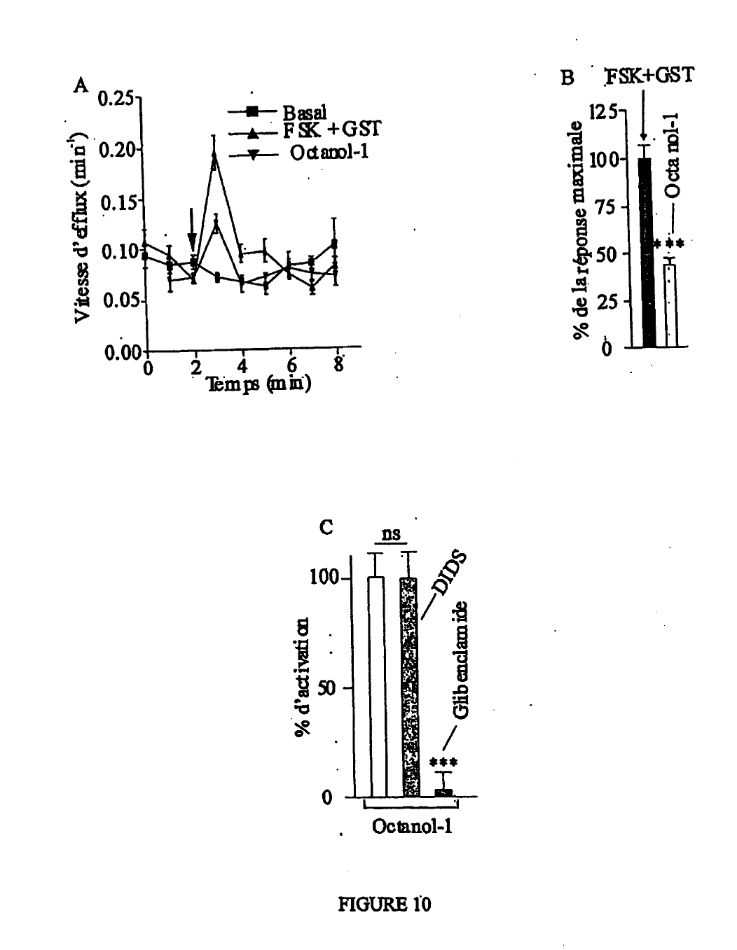

FIG. 10 illustrates: (A) Effect of octan-1-ol (1 mM) (n=8) or of a cocktail of drugs used to give maximum activation of the mutated CFTR-ΔF508 channel (10 μM FSK then 30 μM genistein (GST)) (n=8) on iodide efflux (min⁻¹) as a function of time (min) in JME/CF15 human bronchial epithelial cells extracted from patients suffering from cystic fibrosis. The arrow represents the moment at which the octan-1-ol (1 mM) or the 10 μM FSK+30 μM GST are added. (B) Representation of the percentage activation of CFTR-ΔF508 obtained with octan-1-ol (1 mM) compared with the maximum activity obtained with the 10 μM FSK+30 μM GST cocktail, which is normalized to 100% of the maximum response. (C) Effect of the pretreatment with glibenclamide (100 μM, 1 hour) (n=8) or of the pretreatment with DIDS (500 μM, 1 hour) (n=8) on the maximum response (normalized to 100% activation) obtained with 1 mM of octan-1-ol (t test: *** P<0.001; ns: non significant);

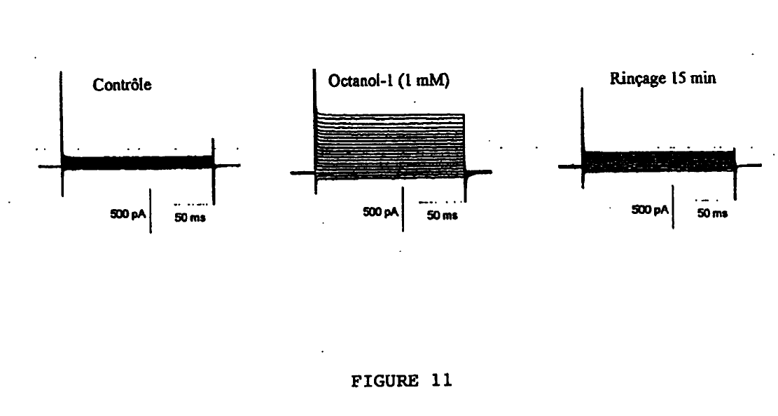

FIG. 11 illustrates the reversibility of the effect of octan-1-ol (1 mM) on the activation of CFTR studied by patch-clamp in the whole cell configuration in a CHO-BQ1 cell. Family of currents evoked in a CHO-BQ1 cell by successive depolarizations between −80 and +15 mV from a maintenance potential of −60 mV and by increments of 5 mV, in the absence (control) and in the presence of octan-1-ol (1 mM) and after washing of the octan-1-ol for 15 min with a physiological saline rinsing medium;

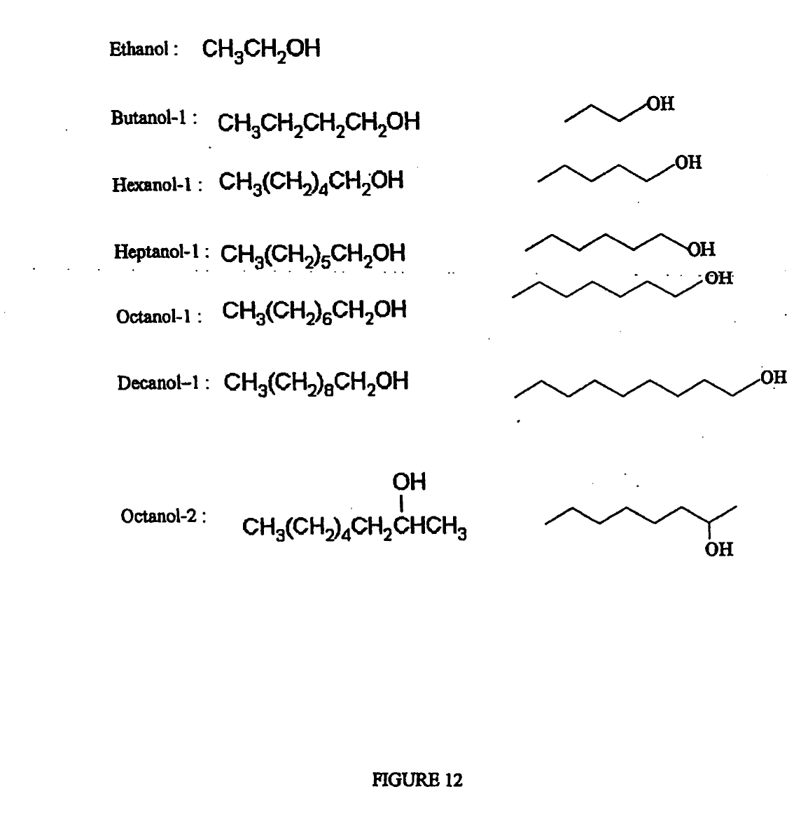

FIG. 12 illustrates the structure of the C₂-C₁₀ n-alkanols.

It should be understood, however, that these examples are given merely by way of illustration of the subject of the invention, of which they in no way constitute a limitation.

### EXAMPLE

### Demonstration of the Properties of N-Alkanols as CFTR Openers or Activators

1) Experimental Methods

1.1) Cells in Culture

The studies on CFTR are, firstly, carried out on CHO cells (Chinese Hamster Ovary cells) that express the recombinant human CFTR protein (CHO-CFTR(+)) (Riordan et al., 1989). The cells are cultured at 37° C. in an incubator saturated with water containing 5% CO₂, in αMEM medium supplemented with fetal calf serum (7.5%), with 2 mM glutamine, with 50 IU/ml of penicillin and with 50 μg/ml of streptomycin. The cells that do not express CFTR are noted CHO-CFTR(−) and are cultured in DMEM/F12 medium under the same conditions as above.

The CFTR studies are also carried out on Calu-3 cells (ATCC No. HTB-55), which are human pulmonary epithelial cells endogenously expressing the CFTR channel. These cells are cultured under the same culture conditions as the CHO cells. The study of the mutated CFTR channel is carried out on JME/CF15 cells, which are epithelial cells extracted from respiratory airways of patients suffering from cystic fibrosis (homozygous ΔF508) (Jefferson et al., 1990). These cells therefore express the ΔF508 mutated CFTR channel. These cells are cultured under the same conditions as above, but the culture medium is supplemented with a hormone mixture containing: adenine (180 μM), insulin (5 μg/ml), transferrin (5 μg/ml), hydrocortisone (1.1 μM), triiodothyronine (2 nM), epinephrine (5.5 μM), epidermal growth factor (1.64 nM).

1.2 Patch-Clamp Technique Applied to the Stud of Cells in Culture

The patch-clamp technique consists in applying a glass pipette or a glass microelectrode to the surface of the cell. By applying slight touch, it is possible to cause the membrane to adhere to the glass. A small piece of membrane (patch) is thus isolated at the end of the pipette. This is the principle of patch-clamp (O. P. Hamill et al., Pflügers Arch., 1981, 391, 85-100; R. Penner, A Practical Guide to Patch Clamping, 1995, In Single Channel Recording, 2nd edition (Eds. B. Sakmann et al.) Plenum Press, New York, 3-30). To do this, the patch-clamp pipette must have a tip of the order of 1 μm in diameter and a resistance of the order of 1-5 MΩ. The resistance of a pipette or of a microelectrode makes it possible to assess the fineness of the tip: the greater the resistance, the finer the tip or the more the electrode is blocked. The diameter of the patch-clamp pipette does not make it possible to penetrate the cell but, on the other hand, it makes it possible effectively to trap a piece of membrane in the tip. Interactions between the membrane and the glass will form, aided by a slight suction or negative pressure in the pipette. The quality of this interaction (or sealing) is also assessed by measuring the resistance between the glass and the membrane. To measure overall currents in the whole cell configuration, a sealing resistance of 1 GΩ is sufficient.

In general, recordings of electric current through a patch containing, for example, a channel are measured. The imposed membrane potential (E_{clamp}) is generally in millivolts. When the channel is closed, the current oscillates weakly around a baseline level (state C). This minute baseline current circulates in the “leaks” between the patch and the end of the pipette. When the channel opens (state O), the current jumps to another level, and then returns to the baseline level when the channel closes, and so on.

The measurements can be carried out in one of the following configurations: cell-attached, whole-cell, inside-out patch or outside-out patch.

The patch-clamp experiments are carried out on confluent cells.

More specifically, culture dishes (cell support) are placed in an experimentation cell (volume 1 ml) on the platform of an inverted microscope (Nikon) equipped with phase-contrast lighting. The whole-cell configuration is used for recording the cell currents (Hamill et al., 1981). The experiments are carried out at ambient temperature (20-22° C.). The currents are amplified with an Axopatch 200B amplifier (Axon Instrument Ltd) having a 2-5 kHz low-pass filter (Bessel 6-pole filter), and recorded on the hard disk of a PC after digitization at 10-25 kHz. The pipettes are produced from glass tubes 1 mm in diameter (Clark Electromedical Instrument) in four steps with a horizontal drawing device (Bruwn Flaming 97, CA).

The pipettes, filled with an intracellular solution containing, in mM: 60 KCl; 80 NMDG (N-methyl-G-glucamine); 10 HEPES; 5 EGTA; 1 CaCl₂; 4 MgATP; 0.2 Na₃GTP; pH 7.4, titrated with KOH), having a resistance of 5 MΩ. The potentials are expressed as the difference between the potential of the patch electrode and that of the bath. In the whole-cell configuration, they represent the membrane potential of the cell. The junction potentials that form between the recording electrode and the extracellular medium are eliminated before the contact of the electrode with the cell. The chlorine current inversion potential is obtained from the Nernst equation (Eᵣₑᵥ=(RT/F)log([Cl]ᵢ/[Cl]ₑ)); i and e are the intracellular and extracellular ion concentration, and R, T and F have their usual meaning. The current-voltage relationships in the stationary state are determined using slow voltage ramps (20 mV/.s) under an imposed voltage condition.

The extracellular recording solution consists of (in mM): 110 NaCl; 23 NaHCO₃; 3 KCl; 1.2 MgCl₂; 2 CaCl₂; 5 HEPES; 11 D-glucose; gassed with 5% CO₂-95% O₂; pH 7.4.

1.3) Measurement of Radioactive Tracer Fluxes Applied to the Study of Epithelial Cells in Culture

Since the CFTR channel is permeable to halides (Br⁻>Cl>I⁻>F⁻), the measurement of ¹²⁵I radioactive iodide efflux proved to be an effective technique for measuring the activity of the CFTR channel (Chang et al., 1998). This technique makes it possible to follow the kinetics of exit of the ¹²⁵I radioactive iodide. The cells are cultured in 24-well plates with a dilution to 1/10 after passage. On the fourth day of culture, the drugs to be tested are dissolved in solution according to the desired concentration, at 37° C., in medium B, at pH 7.4, containing, in mM: 137 NaCl, 5.36 KCl, 0.8 mM MgCl₂, 1.8 mM CaCl₂, 5.5 glucose and 10 HEPES-NaOH. The wells are washed 4 times with 500 μl of medium B. The solution is subsequently replaced with 500 μl of loading solution containing 1 μM KI and 0.5 μCi of ¹²⁵INa/ml for 30 min. The kinetics of exit of ¹²⁵I are determined after having eliminated the loading solution and washed the wells 4 times with 500 μl of medium B. For the ¹²⁵I exit kinetics, 500 μl of medium B containing or not containing the molecules to be tested are incubated for 30 sec in the wells and recovered in a hemolysis tube, so as to be replaced with 500 μl of medium B containing or not containing the molecules to be tested. The operation is repeated every 30 s for 2 to 6 min. At the end of the efflux, the intracellular ions are extracted by adding 1 ml of trichloroacetic acid (7.5%) to the cell layer. All the samples are counted in a gamma counter (Kontron). The precipitated proteins are solubilized in 0.1N NaOH and quantified using a calorimetric test.

1.4) Data Analysis

The tracer contained in the cell layer at the beginning of the efflux is calculated as the sum of the samples and of the extracts counted. The efflux curves are constructed by expressing the percentage of the content remaining in the cell layer (I %) with respect to time. The stimulated or nonstimulated efflux rate constants (k, min⁻¹) are determined by smoothing the efflux curves to a monoexponential function I %=100*exp(−kt) using a linear regression of the neperian logarithm of the data, k is used to calculate the iodide released into the medium with respect to time. The hypothesis is made that, in the presence of a stimulator, the efflux is the sum of two iodide effluxes occurring in parallel: a basal efflux and a stimulated efflux characterized, respectively, by the constants k_{b} and kₛ. The total net efflux is then described by the equation Iₜ^{%}=100*(1−exp(−kₜt)) where kₜ is the sum of k_{b} and kₛ. Finally, kₛ calculated as kₜ−k_{b} is used to establish a dose-response relationship for antagonists. The data is expressed as means ±SD, and the t-test is used to determine the significances. The concentration-response curves for agonists or for antagonists are smooth using the hyperbolic equation Y=Yₘₐₓ*X/(EC_{5C}+X), where Y is the response. Y begins at the basal level and progresses to the plateau (Yₘₐₓ), X is the concentration and the half-effect values (EC are calculated using GraphPad Prism v3.0 (GraphPad software)).

1.5) Assaying of Total Intracellular cAMP

The CHO cells are cultured for four days in a 24-well culture plate. On the fourth day of culture, each well is rinsed twice with 500 μl of medium B, and 500 μl of this buffer containing the molecule to be tested are added to each well. After incubation at 37° C. for 5 min, the reaction is stopped by adding a cell lysis buffer. The cell lysis is verified with Trypan blue. The amount of cAMP contained in the cells is determined using the Enzyme Immuno Assay kit (Amersham Biotechnology). The cAMP level is expressed in pmol/well ±SD.

1.6) Cell Imaging and Measurement of Intracellular Ca²⁺ Concentration ([Ca²⁺]ᵢ)

The Ca²⁺ measurements are carried out in the presence of fura-2 (impermeant fluorescent probe) which binds Ca²⁻. The cells are incubated in serum-free DMEM/F12 culture medium in the presence of the permeant form of fura-2 (fura-2/AM, 2.5 μM) for 1 h at 37° C. The cells, placed on the platform of an epifluorescent microscope (Olympus) (20× objective), are perfused with the solutions to be tested (e.g. ATP, octanol). They are sequentially illuminated at 340 nm and 380 nm and the fluorescence emitted (F) is measured at 510 nm. It is detected via a 12-bit CCD camera (Sony) connected to a data processing computer unit (TILL Photonics). The intracellular Ca²⁺ concentration [Ca²⁺]ᵢ is calculated using the Grynckiewicz equation (Grynckiewicz et al., 1985): [Ca²⁺]ᵢ=K_{di *(R-R}ₘᵢₙ/Rₘₐₓ−R), based on the ratiometric analysis R (340/380 nm) of the fluorescence (TILL Vision); Rₘₐₓ and Rₘᵢₙ denote the ratio of fluorescence measured in the presence or in the absence of Ca²⁺.

2) Results

The n-alkanol concentrations used range from 0.1 to 10 mM. These concentrations represent final proportions (v/v) of from 0.001% (for 0.1 mM of alcohols) to a maximum of 0.1% (for 10 mM of alcohols).

2.1) Octanol Specifically Activates the CFTR Channel

The molecules tested are n-alkanols, and in particular octan-1-ol, which were tested for their ability to activate the CFTR channel.

The screening of the molecules as CFTR channel openers was carried out by measuring their effect on ¹²⁵I radioactive iodide efflux and on transmembrane chloride currents. These data were supplemented by measurement of the intracellular cyclic AMP (cAMP) level and of variations thereof in various experimental situations.

Evaluation of the effect of CFTR activation by the n-alkanols is first of all carried out on the CHO-CFTR(+) cell line. CHO-CFTR(−) cells that do not express the CFTR protein were used as control cells. A23187 (2 μM) (a calcium ionophore) has no effect on ¹²⁵I efflux, both in CHO-CFTR(+) and CHO-CFTR(−) cells, showing that there are no intracellular calcium-dependent Cl⁻ channels (Chappe et al., 1998). Since the CFTR channel is mainly regulated by protein kinases A, the control experiments made use of FSK for stimulating the intracellular cAMP level.

FIG. 1A shows an activation of CFTR obtained by application either of 1 μM of FSK (FSK) or of octan-1-ol (1 mM) or a combined application of FSK (1 μM) and of octan-1-ol (1 mM) to CHO-CFTR(+) cells. The activation of the CFTR channel, measured by the ¹²⁵I efflux, induces an increase in the amplitude of the iodide efflux (expressed as % of ¹²⁵I released into the medium) and in the rate of exit of ¹²⁵I.

In FIG. 1B, the control experiments for evaluating the specificity of the molecules tested on the activity of the CFTR channel were carried out on CHO-CFTR(−) cells, in the presence or absence of activators (1 μM FSK, 1 mM octan-1-ol). In these CHO-CFTR(−) cells, octan-1-ol (0.1 to 5 mM) and FSK (5 μM) do not significantly modify the basal level of ¹²⁵I efflux (FIG. 1C).

The activating effect of octan-1-ol and also of FSK (1 μM) or of a combined application of FSK and octan-1-ol on CFTR is completely inhibited by the addition of glibenclamide (100 μM), which is commonly used to specifically inhibit the CFTR channel (FIG. 1D). These results confirm the specificity of octan-1-ol on the activity of CFTR.

The CHO-CFTR(+) cells are subsequently stimulated with increasing concentrations of FSK or of octan-1-ol, and the ¹²⁵I efflux rate constants are measured. FIG. 2 represents a dose-response curve for octan-1-ol (0.1 to 5 mM) or for FSK (0.1 to 5 μM) on the activation of CFTR. It can be seen in FIG. 2 that the activation of CFTR by FSK or by octan-1-ol is dependent on the concentration, with an EC₅₀ of approximately 0.5 μM for FSK and of 0.5 mM for octan-1-ol. The effects of octan-1-ol on the activation of CFTR can be observed for octan-1-ol concentrations of 0.3 mM to 5 mM, with a plateau reached at 1 mM and a half-activation dose of 0.5 mM.

Similarly, the activation of CFTR by FSK or octan-1-ol is observed by a patch-clamp in the whole-cell configuration. FIGS. 3A and 3B show, respectively, that FSK (1 μM) and octan-1-ol (1 mM) produce an approximately 10-fold increase in membrane conductance compared with the control. The reversion potential of the FSK- or octan-1-ol-induced current is 1±0.6 mV, showing that Cl⁻ is the main ion that contributes to this current. This FSK- or octan-1-ol-induced current is completely inhibited by the application of glibenclamide, showing that it is indeed the CFTR channel that is involved in the current induced either by FSK or by octan-1-ol. FIG. 3B shows that application of octan-1-ol alone (1 mM), i.e. without FSK, induces a full activation of the CFTR channel.

2.2) Octan-1-ol Specifically Stimulates the CFTR Channel in Human Bronchial Epithelial Cells (Calu-3)

The ability of octan-1-ol to stimulate CFTR in a human bronchial epithelial cell line (Calu-3) was also tested. As shown in FIG. 9A, octan-1-ol activates the exit of iodide in Calu-3 cells. This octan-1-ol-activated efflux is strongly blocked by treatment (1 hour) with glibenclamide (100 μM), a CFTR channel inhibitor, whereas treatment (1 hour) with 500 μM of DIDS (4,4′-diisothiocyanatostilbene-2,2′-disulfonic acid), used to block Cl⁻ channels except the CFTR channel which is insensitive thereto, has no effect (FIG. 9B). All these results show that octan-1-ol specifically activates the human CFTR channel endogenously expressed in Calu-3 human bronchial epithelial cells. Furthermore, as observed in the CHO-CFTR(+) cells (see FIG. 1), octan-1-ol activates the human CFTR channel in a dose-dependent manner (0.1 to 10 mM) in Calu-3 cells (FIGS. 9C and D). Finally, a dose-response curve for octan-1-ol produced in the presence of FSK (1 μM) shifts to the left the dose-response curve for octan-1-ol, indicating a potentiation by forskolin of the activation of CFTR by octan-1-ol. In addition, the activation of the CFTR channel by octan-1-ol for concentrations greater than 0.5 mM is greater than that obtained with FSK (1 μM), since octan-1-ol potentiates the activity of the CFTR channel stimulated by FSK (1 μM) (FIGS. 9C and D).

2.3) Octan-1-ol Specifically Activates the ΔF508 Mutated CFTR Channel in Epithelial Cells of Pulmonary Origin (CF15) from ΔF508 Homozygous Patients Suffering from Cystic Fibrosis

The ability of octan-1-ol to activate the ΔF508 mutated CFTR channel was tested. The ΔF508 mutation is found in more than 70% of patients suffering from cystic fibrosis. A large majority of the ΔF508 mutated CFTR channel is degraded by the ubiquitin-proteasome system inside the cell, and only a very small amount of mutated channel reaches the surface of the membrane, where it can be activated. JME/CF15 human bronchial epithelial cells extracted from patients suffering from cystic fibrosis and homozygotes for the ΔF508 mutation were used. The MPB-91 molecule, known to target a certain number of CFTR-ΔF508 channels to the plasma membrane (Dormer et al., 2001), was also used. The ΔF508 mutated CFTR channels, present at the plasma membrane, were stimulated with octan-1-ol (1 mM). FIG. 10A shows that octan-1-ol specifically activates the ΔF508 mutated CFTR channel. A cocktail of stimulators (10 μM FSK+30 μM genistein) makes it possible to obtain the maximum activity for the mutated CFTR-ΔF508 channel. Octan-1-ol (1 mM) is capable, by itself, of activating approximately 50% of the maximum activity of the mutated CFTR-ΔF508 channel (FIG. 10B). This CFTR-ΔF508 activation is inhibited by glibenclamide (100 μM) whereas it is insensitive to DIDS (500 μM) (FIG. 10C), demonstrating that octan-1-ol specifically stimulates the ΔF508 mutated CFTR channel. Furthermore, octan-1-ol has no effect on the basal level, when the mutated channel is not present at the plasma membrane, showing that it does not activate other chloride conductances and that it is indeed specific for the CFTR channel. All these results demonstrate that octan-1-ol is capable of activating the ΔF508 mutated CFTR channel in human pulmonary epithelial cells from patients suffering from cystic fibrosis. Octan-1-ol is thus of great interest for envisioning a pharmacotherapeutic treatment for cystic fibrosis.

2.4) The Activating Effect of Octan-1-ol on CFTR is Reversible

Finally, we examined, by means of the whole-cell configuration patch-clamp technique on CHO-BQ1 cells that express the human CFTR chanel, whether the effect of octan-1-ol (1 mM) on CFTR channels was reversible. As indicated in FIG. 11, it is seen that octan-1-ol (1 mM) brings about an increase in current due to the activation of CFTR. After washing of octan-1-ol (1 mM) with a physiological saline solution for 15 min, the CFTR channel activation disappears, indicating that the activating effect of octan-1-ol on CFTR is reversible.

2.5) Long (C₆ to C₁₀) Hydrocarbon-Chain n-Alkanols Activate the CFTR Channel (See FIG. 12 for the Structure of the n-Alkanols)

The effect of n-alkanols other than octan-1-ol on the activation of CFTR was also tested. FIG. 4A shows that the use of n-alkanols having hydrocarbon chain lengths greater than or equal to those of hexan-1-ol (C6) up to decan-1-ol (C10) significantly activates the CFTR channel. The activation of CFTR increases as a function of the length of the hydrocarbon chain (i.e. as a function of the hydrophobicity) of the alcohol. FIG. 4 shows an increase in activation of CFTR following the application of hexan-1-ol (C6), of heptan-1-ol (C7), of octan-1-ol (C8), of decan-1-ol (C10). For n-alkanols having short hydrocarbon chains (methanol, butan-1-ol), the ¹²⁵I efflux is not significantly different from the unstimulated efflux, indicating that ethanol and butanol do not activate the CFTR channel.

In FIG. 4B, it can be seen that octan-2-ol also activates the CFTR protein. This shows that the position of the OH radical on the molecule in the 1-position or 2-position is not essential for activation of the CFTR channel.

2.6) The Activation of CFTR by n-Alkanols is not Due to Cellular Uncoupling

Octanol and the other n-alkanols can modify cellular uncoupling due to gap junctions. Such uncoupling is demonstrated in CHO cells, by measuring the calcium response induced by application of ATP. To do this, a molecule completely different from n-alkanols but known to uncouple cells, 18-alpha glycerrhetinic acid (α-GA), was used. FIGS. 5A-C show that the application of α-GA (10 to 100 μM) or else octan-1-ol (1 mM) clearly uncouples the cells, as shown by the ATP-induced calcium response. However, no effect of (α-GA) on the activity of the CFTR channel is observed (FIG. 6). The activation of CFTR by n-alkanols is not therefore due to their cellular uncoupling property.

2.7) Protein Kinase A Activity is Required for CFTR Activation by Octan-1-ol

Phosphorylation of the CFTR channel, in particular by protein kinase A (PKA), has been shown to be required for the function and the activation of the channel. The activation of the CFTR channel by the n-alkanols is inhibited by treatment with H-89 (30 μM), used to inhibit PKAs (FIG. 5A), which shows that constitutive phosphorylation of the CFTR channel is required for its activation by octan-1-ol.

Recent studies have shown that phosphorylation of CFTR by protein kinase C (PKC) could be a prerequisite for CFTR activation. Octanol has been shown to be capable of activating certain PKC subtypes. A powerful inhibitor of PKCs in the presence of octan-1-ol was therefore used. Under these conditions, the activation of CFTR by octan-1-ol is not inhibited (FIG. 7). These results show that octan-1-ol clearly activates CFTR via a PKC-independent mechanism.

Octanol and n-alkanols can interact directly with the CFTR channel at the hydrophobic sites of the protein, in order to induce a conformational modification favorable to its activation.

The n-alkanols do not induce any increase in cAMP, and the activation of CFTR by the n-alkanols is not therefore due to an increase in the cAMP level induced by the n-alkanols.

The literature indicates that long-chain n-alkanols are not potential activators of adenylate cyclase and therefore of the intracellular cAMP level, but would rather have an inhibitory effect. FIG. 8 gives the intracellular cAMP level in the CHO-CFTR(+) cell, measured after 5 min in the presence of 5 μM or 1 μM of FSK (activator of the enzyme for cAMP synthesis; adenylate cyclase), or of 1 mM of octan-1-ol, of hexan-1-ol or of ethanol. While 1 μM or 5 μM FSK significantly increases the cAMP level, neither octan-1-ol, hexan-1-ol or ethanol modifies the basal cAMP level. When applied alone, octan-1-ol triggers activation of the CFTR channel without increasing the cAMP level. These results show that octanol and the other C₆ to C₁₀ long-hydrocarbon-chain n-alkanols stimulate the CFTR channel via a cAMP-pathway-independent pathway.

### BIBLIOGRAPHIC REFERENCES

Becq et al., (1994). PNAS. 9160-9164
Becq et al., (1999). J. Biol.Chem. 274: 27415-27425
Chang et al., (1998). Methods Enzymol. 92: 616-629
Cutting et al., (1990). Nature. 346: 366-369
Dalemans et al., (1991). Nature. 354: 526-528
Dormer et al., (2001), J. Cell Sci., 114: 4073-4081
Drumm et al., (1991). Science. 254:1797-1799
Eidelman et al., (1992). PNAS 89: 5562-5566
Gregory et at., (1991). Mol Cell Bio 11: 3886-38931
Gribkoff et al., (1994). J. Biol. Chem. 269: 10983-10986
Griesenbach et al., (1999). Thorax. 54: S19-S23
Hamill et al., (1981), Pflugers Arch. 391: 85-100
Marzalec et al., (1994). J. Pharmacol. Exp. Ther. 269: 157-163
Mascia et at., (2000). PNAS 97: 9305-9310
Miller & Dallas, (1973). Arch. Intern. Med. 131: 148-155
Nakahiro et al., (1999), J. Pharmacol. Exp. Ther. 259: 235-240
Narahashi et al., (1998). Toxicolletter. 100-101: 185-191
Reddy et al., (1999). Nature. 402: 301-304
Riordan et al., (1989). Science. 245: 1066-1073
Sakihara et al., (2000). Anesthesiology. 96: 428-437
Schwiebert et al., (1995). Cell. 81: 1063-1073
Sheppard et al., (1993). Nature. 362: 160-164
Tabcharani et al., (1991). Nature. 352: 628-631
Tsui & Buchwald, (1991). Advances in human genetic 20: 153-266
Wei et al., (1999) Pfügers Arch. 438: 635-641
Weingart & Bukauskas, (1998). Pfügers Arch. 435: 310-319

1. The use of C₆-C₁₀ linear, possibly branched, or cyclic hydrocarbon-chain n-alkanols, for preparing a medicinal product for use in the treatment of pathology related to CFTR chloride channel disorders in humans or animals.

2. The use as claimed in claim 1, wherein said n-alkanols are linear, possibly branched, hydrocarbon-chain n-alkanols in which the OH group is in the 1-position (primary alcohol) or in the 2-position (secondary alcohol).

3. The use as claimed in claim 1, wherein said n-alkanols are cyclic hydrocarbon-chain n-alkanols carrying one or more alcohol groups.

4. The use as claimed in claim 1, wherein said pathologies are selected from the group consisting of cystic fibrosis, atypical cystic fibrosis, and obstructions of the bronchial tracts or of the digestive tracts.

5. The use as claimed in claim 1, wherein said n-alkanols are provided in a form suitable for intranasal or buccal administration.

6. The use as claimed in claim 5, wherein said n-alkanols are provided in a liquid form, for administration in the form of an aerosol or in the form of a nebulized material.

7. The use as claimed in claim 6, wherein said n-alkanols are combined with at least one pharmaceutically acceptable carrier appropriate for said intranasal or buccal administration.

8. The use as claimed in claim 1, said n-alkanols are administered at a concentration of between 0.001% and 0.1% (v/v), corresponding to a value of between 10 and 1000 ppm (parts per million), i.e. from 10 mg/kg to 1 g/kg.
